(19) United States
(12) Patent Application Publication
(10) Pub. No.: US 2024/0384202 A1
(43) Pub. Date: Nov. 21, 2024

(54) END-GROUP CAPPED, BIO-BASED LOW FOAMING SURFACE ACTIVE AGENTS

(71) Applicant: CLARIANT INTERNATIONAL LTD, Muttenz (CH)

(72) Inventors: Dirk LEINWEBER, Kelkheim (DE); Goswinus DE KRUIJFF, Muehldorf am Inn (DE); Johannes BOOKHOLD, Neu-Anspach (DE); Tobias PLATEN, Hochheim am Main (DE); Juan SARRIA, Hattersheim am Main (DE); Xiaoqiang GUO, Frankfurt am Main (DE); Christine OBERBILLIG, Bischofsheim (DE)

(21) Appl. No.: 18/696,533

(22) PCT Filed: Sep. 29, 2022
(86) PCT No.: PCT/EP2022/077185

§ 371 Date: Mar. 28, 2024

(30) Foreign Application Priority Data
Oct. 1, 2021 (EP) 21200431.1

(51) Int. Cl. C11D 1/72; C07C 67/02; C07C 67/08; C07C 69/716; C11D 3/386

(52) U.S. Cl. CPC C11D 1/721; C07C 67/02; C07C 67/08; C07C 69/716; C11D 3/38618

(57) ABSTRACT

Sustainable end group-capped surface active agents of the formula

R¹—O— [symbol] (CH₂CH₂O)ₓ(CHR³CHR⁴O)_{y} [symbol] —C(O)—Z—C(O)—R² (I)

which are based on esterified fatty alcohol polyglycol ethers with a ketocarboxylic acid, in particular levulinic acid, a process for their preparation and their use as low foaming surface active agents in detergent compositions and as rinse aid in machine dishwashing is described.

The invention relates to sustainable end group-capped, low foaming surface active agents based on esterified fatty alcohol polyglycol ethers, a process for their preparation according to green chemical principles and to their use as low foaming surfactants in various applications. Furthermore, the invention relates to detergent compositions comprising these surface active agents and the use thereof as rinse aid in machine dishwashing.

The foam development associated with the use of surface-active substances represents a serious problem for many technical processes. Considering that surfactants lower the surface tension between water and air they inherently are prone to foaming which is a desired phenomenon in applications like hand dish washing liquids or shower gels.

In contrast, foam formation needs to be avoided in several consumer and industrial applications to ensure maximum anticipated performance profiles. In applications like automatic dishwashing, metal working, laundry and industrial detergents, crop protection, pulp and paper manufacturing, textile processing and pigment dispersions, low foaming profiles of surfactants are strived for and very beneficial.

Prior art describes various strategies towards low foaming surface active agents.

Fatty alcohol polyglycol ethers that have been end-capped have proven their value in recent years. These so-called “mixed ethers” are obtained via Williamson's ether synthesis by reacting fatty alcohol polyglycol ethers with alkyl halides (DE 37 44 525 C1). However, the production of these substances has two disadvantages: on the one hand, the reaction takes place in the presence of stoichiometric amounts of an alkali metal hydroxide and is accordingly associated with a high salt accumulation, on the other hand, working with alkyl halides makes high demands on occupational safety.

From DE 40 41 184 A1 esters of C₁-C₂₂-alkanols, in particular C₈-C₁₆-alkanols, with di- and polycarbonic acids (e.g. citric acid) are also known which act as defoamers. For many applications, however, these products are not sufficiently water-soluble.

DE 1 243 312 B describes end-capped low foaming surfactants which are substituted polyglycol ethers obtained by esterifying C₈-C₂₄-alkyl alcohol polyglycol ethers with C₁-C₁₀-alkyl carboxylic acids.

WO 94/03251 A1 also describes end-capped low foaming surfactants which are obtained by esterifying fatty (C₁-C₂₂-alkyl)-alcohol polyglycol ethers with C₆-C₂₂-alkyl carboxylic acids in presence of acidic catalysts.

None of the afore-mentioned end-capping carboxylic acids contains a keto group, nor is a completely bio-based low foaming surface active agent obtained from (partially) second generation feedstocks.

There is a need for low foaming surface active agents which are sustainable and producible according to green chemical principles which can be used for foam regulation in the afore-mentioned applications.

Therefore, the object of the present invention is to provide low foaming surface active agents which are sustainable and producible according to green chemical principles which can be used for foam regulation in the afore-mentioned applications, in particular as detergents for machine dishwashing, laundry detergents and industrial cleaners.

Surprisingly, it was found that a short chain (preferably bio-based) keto carboxylic acid or its ester when used as end-cap for fatty alcohol polyglycol ethers results in very effective, low foaming surface active agents, which are sustainable and producible according to green chemical principles.

One aspect of the invention relates to a surface active agent of the formula (I)

R¹—O— [symbol] (CH₂CH₂O)ₓ(CHR³CHR⁴O)_{y} [symbol] —C(O)—Z—C(O)—R² (I)
wherein
R¹ is a linear or branched alkyl group having 8 to 30 carbon atoms, preferably 8 to 22 carbon atoms, or a linear or branched alkenyl group having one or more, often 1 to 3, double bonds and 8 to 30 carbon atoms, preferably 8 to 22 carbon atoms;
X is an integer from 1 to 25;
y is an integer from 0 to 10;
R³, R⁴ are chosen such that either R³ or R⁴ is hydrogen and the other substituent is a methyl group, preferably R³ is hydrogen and R⁴ is a methyl group;
Z is a linear or branched alkylene having 1 to 3 carbon atoms,
R² is a linear or branched alkyl group having 1 to 3 carbon atoms, preferably methyl or ethyl, more preferably methyl;
where if y is ≠0, the structure units-CH₂CH₂O— and —CHR³CHR⁴O—, in particular
—CH₂CH₂O— and —CH₂CH(CH₃)O—, may be arranged in any desired order, preferably in random, gradient-like or block-like manner, more preferably in block-like manner, and x is greater than y.

Preferably in the surface active agent of formula (I), x is an integer from 1 to 20, more preferably from 2 to 20, even more preferably from 2 to 15, particularly preferably from 5 to 10, extraordinarily preferably from 8 to 10.

Preferably in the surface active agent of formula (I), y is an integer from 0 to 5, more preferably from 0 to 3, most preferably 0.

In case y is ≠0, the (molar) ratio of x to y is higher than 1, preferably at least 2, more preferably at least 3, and even more preferably at least 4.

Further preferred is a surface active agent of the formula (I) comprising structure units [symbol] (CH₂CH₂O)ₓ(CHR³CHR⁴O)_{y} [symbol] -derived from bio-ethylene oxide and, if present, derived from bio-propylene oxide.

Most preferably in the surface active agent of the formula (I), y=0 and the ethylene oxide (CH₂CH₂O)-structure units are derived from bio-ethylene oxide.

Bio-ethylene oxide and/or bio-propylene-oxide means an ethylene oxide and/or propylene-oxide obtained from renewable resources.

Preferably in the surface active agent of the formula (I) Z is a linear or branched alkylene having 1 to 3 carbon atoms, preferably methylene, ethylene (—CH₂—CH₂—) or isopropylene (—CH₂—CH(CH₃)— or —CH(CH₃)—CH₂—), more preferably methylene or ethylene, most preferably ethylene.

More preferably in formula (I) Z is methylene, ethylene or isopropylene, preferably methylene or ethylene, and R² is methyl or ethyl, preferably methyl.

In particular preferred in formula (I) R² is methyl and Z is ethylene.

Even more preferred the structure unit —C(O)—Z—C(O)—R² of formula (I) is a residue derived from a bio-based keto carboxylic acid or bio-based keto carboxylic acid ester, in particular derived from levulinic acid or a levulinic acid C₁ to C₄-alkyl ester, especially levulinic acid methyl ester.

In the context of the invention a bio-based keto carboxylic acid means that it is produced from crops and plant based material. Preferably, the keto carboxylic acid is produced from second generation feedstocks including but not limited to second generation waste feedstocks. “Second generation feedstock” as used herein means the use of non-food (not suitable for consumption as food for humans or animals) crops, biomass and wastes (including first generation biomass waste streams) as feedstocks.

Levulinic acid (4-oxo pentane carboxylic acid) is preferably employed as a second-generation bio-based compound, which stems from cellulosic feedstocks like wheat straw, lignocellulose or sugarcane bagasse.

Preferably in the surface active agent of the formula (I) R¹ is a linear or branched alkyl group having 8 to 22 carbon atoms, more preferably 8 to 18 carbon atoms, even more preferably 16 to 18 carbon atoms, or a linear or branched alkenyl group having one or more, often 1 to 3, double bonds and 8 to 22 carbon atoms, more preferably 8 to 18 carbon atoms, even more preferably 16 to 18 carbon atoms.

Examples for the alkyl- and alkenyl-groups of R¹ of formula (I) are, e.g. the alkyl and alkenyl groups of the following alcohols R¹—OH: 1-octanol (caprylic alcohol), 2-ethylhexanol, 1-nonanol (pelargonic alcohol), 1-decanol (capric alcohol), 1-undecanol, 1-dodecanol (lauryl alcohol), 1-tridecanol, isotridecanol, 1-tetradecanol (myristyl alcohol), 1-pentadecanol, 1-hexadecanol (cetyl alcohol), cis-9-hexadecene-1-ol (palmitoleyl alcohol), 1-heptadecanol, 1-octadecanol (stearyl alcohol), cetearyl alcohol, 16-methylheptadecan-1-ol (Isostearyl alcohol), 9E-octadecene-1-ol (elaidyl alcohol), cis-9-octadecene-1-ol (oleyl alcohol), oleyl cetyl alcohol (i.e., a mixture of oleyl alcohol and cetyl alcohol), 9Z,12Z-octadecadien-1-ol (linoleyl alcohol), 9E,12E-octadecadien-1-ol (Elaidolinoleyl alcohol), 9Z,12Z,15Z-octadecatrien-1-ol (linolenyl alcohol), 9E, 12E,15E-octadecatriene-1-ol (elaidolinolenyl alcohol), 1-nonadecanol, 1-eicosanol (arachidyl alcohol), (Z)-icos-9-en-1-ol (gadoleyl alcohol), 1-heneicosanol, 1-docosanol (behenyl alcohol), cis-13-docosen-1-ol (erucyl alcohol), 1-tetracosanol (lignoceryl alcohol), 1-hexacosanol (ceryl alcohol), 1-octacosanol (montanyl alcohol) and 1-triacontanol (myricyl alcohol) or mixtures thereof.

Preferred among the afore-mentioned alcohols R¹—OH are natural fatty acid alcohols, preferably obtained from biomass, generally from first generation feedstocks, i.e. edible oil feedstocks.

Particularly preferred as alcohol R¹—OH is lauryl alcohol, oleyl alcohol or cocoyl alcohol. Cocoyl alcohol generally is a mixture of C₈ to C₁₈ fatty alcohols.

Accordingly, particularly preferred the alkyl or alkenyl group R¹ is derived from lauryl alcohol, oleyl alcohol or cocoyl alcohol.

Particularly preferred are surface active agents of the formula (I1)

R¹—O [symbol] (CH₂CH₂O)ₓ₁(CHR³CHR⁴O)_{y1} [symbol] —C(O)—Z—C(O)—R² (I1)
wherein
R¹ is a linear or branched alkyl group having 8 to 30 carbon atoms, preferably 8 to 22 carbon atoms, more preferably 8 to 18 carbon atoms, even more preferably 16 to 18 carbon atoms or a linear or branched alkenyl group having one or more, preferably 1 to 3, double bonds and 8 to 30 carbon atoms, preferably 8 to 22 carbon atoms, more preferably 8 to 18 carbon atoms, even more preferably 16 to 18 carbon atoms;
x1 is, based on a molar average, a number from 1 to 25, preferably from 1 to 20, more preferably from 2 to 20, even more preferably from 2 to 15, particularly preferably from 5 to 10, extraordinarily preferably from 8 to 10;
y1 is, based on a molar average, a number from 0 to 10, preferably from 0 to 5, more preferably from 0 to 3, even more preferably 0;
R³, R⁴ are chosen such that either R³ or R⁴ is hydrogen and the other substituent is a methyl group, preferably R³ is hydrogen and R⁴ is a methyl group;
Z is a linear or branched alkylene having 1 to 3 carbon atoms, preferably ethylene (CH₂CH₂);
R² is a linear or branched alkyl group having 1 to 3 carbon atoms, preferably methyl or ethyl, more preferably methyl;
where if y1 is ≠0, the structure units-CH₂CH₂O— and —CHR³CHR⁴O—, in particular
—CH₂CH₂O— and —CH₂CH(CH₃)O—, may be arranged in any desired order, preferably in random, gradient-like or block-like manner, more preferably in block-like manner, and x1 is greater than y1.

Extraordinarily preferably the surface active agent of the formula (I) is a compound of the formula (Ia)

R¹—O [symbol] (CH₂CH₂O)ₓ [symbol] —C(O)—CH₂CH₂—C(O)—CH₃ (Ia)

wherein R¹ and x are as defined above.

Generally the structure unit C(O)—CH₂CH₂—C(O)—CH₃ of formula (Ia) is derived from levulinic acid or a levulinic acid C₁ to C₄-alkyl ester, especially levulinic acid methylester.

Preferably in formula (Ia) x is an integer from 2 to 20, more preferably from 2 to 15, even more preferably from 5 to 10, particularly preferably from 8 to 10, and R¹ is a linear or branched alkyl group having 8 to 22 carbon atoms, more preferably 8 to 18 carbon atoms, even more preferably 16 to 18 carbon atoms, or a linear or branched alkenyl group having one or more, often 1 to 3, double bonds and 8 to 22 carbon atoms, preferably 8 to 18 carbon atoms, even more preferably 16 to 18 carbon atoms.

Furthermore extraordinarily preferred surface active agents are those of the formula (Ia1)

R¹—OØ(CH₂CH₂O)ₓ₁ℏ—C(O)—CH₂CH₂—C(O)—CH₃ (Ia1)
wherein
R¹ is a linear or branched alkyl group having 16 to 18 carbon atoms or a linear or branched alkenyl group having one or more, preferably 1 to 3, double bonds and 16 to 18 carbon atoms, and
x1 is, based on a molar average, a number from 8 to 10.

The invention further relates to a process for the preparation of the surface active agents of the formula (I)

R¹—O [symbol] (CH₂CH₂O)ₓ(CHR³CHR⁴O)_{y} [symbol] —C(O)—Z—C(O)—R² (I)
wherein
a compound of the formula (II)

R¹—O [symbol] (CH₂CH₂O)ₓ(CHR³CHR⁴O)_{y} [symbol] —H (II)
is esterified in presence of at least one acidic esterification catalyst
or transesterified in presence of at least one transesterification catalyst with
a compound of the formula (III)

R⁵O—C(O)—Z—C(O)—R² (III),
wherein in the formulae R¹, R², R³, R⁴, x, y and Z are as defined in formula (I) above, and R⁵ denotes a C₁ to C₄-alkyl group, preferably ethyl or methyl, more preferably methyl, or hydrogen (H).

Preferably the compound of formula (II) is a fatty alcohol polyglycolether wherein R¹ is a linear or branched alkyl group having 8 to 22 carbon atoms, more preferably 8 to 18 carbon atoms, even more preferably 16 to 18 carbon atoms, or a linear or branched alkenyl group having one or more, often 1 to 3, double bonds and 8 to 22 carbon atoms, preferably 8 to 18 carbon atoms, and more preferably 16 to 18 carbon atoms

More preferably the compound of formula (II) is a fatty alcohol polyglycolether, wherein y=0 and x=2 to 15, preferably 5 to 10, more preferably 8 to 10.

Even more preferred is a process for the preparation of the surface active agents of the formula (I1)

R¹—O [symbol] (CH₂CH₂O)ₓ₁(CHR³CHR⁴O)_{y1} [symbol] C(O)—Z—C(O)—R² (I1)
wherein
a compound of the formula (II1)

R¹—O [symbol] (CH₂CH₂O)ₓ₁(CHR³CHR⁴O)_{y1} [symbol] H (II1)
is esterified in presence of at least one acidic esterification catalyst
or transesterified in presence of at least one transesterification catalyst with
a compound of the formula (III)

R⁵O—C(O)—Z—C(O)—R² (III),
wherein in the formulae
R¹ is a linear or branched alkyl group having 8 to 30 carbon atoms, preferably 8 to 22 carbon atoms, more preferably 8 to 18 carbon atoms, even more preferably 16 to 18 carbon atoms or a linear or branched alkenyl group having one or more, preferably 1 to 3, double bonds and 8 to 30 carbon atoms, preferably 8 to 22 carbon atoms, more preferably 8 to 18 carbon atoms, even more preferably 16 to 18 carbon atoms;
x1 is, based on a molar average, a number from 1 to 25, preferably from 1 to 20, more preferably from 2 to 20, even more preferably from 2 to 15, particularly preferably from 5 to 10, extraordinarily preferably from 8 to 10;
y1 is, based on a molar average, a number from 0 to 10, preferably from 0 to 5, more preferably from 0 to 3, even more preferably 0;
R³, R⁴ are chosen such that either R³ or R⁴ is hydrogen and the other substituent is a methyl group, preferably R³ is hydrogen and R⁴ is a methyl group;
Z is a linear or branched alkylene having 1 to 3 carbon atoms, preferably ethylene (CH₂CH₂);
R² is a linear or branched alkyl group having 1 to 3 carbon atoms, preferably methyl or ethyl, more preferably methyl;
R⁵ denotes a C₁ to C₄-alkyl group, preferably ethyl or methyl, more preferably methyl, or hydrogen (H);
where if y1 is ≠0, the structure units-CH₂CH₂O— and —CHR³CHR⁴O—, in particular-CH₂CH₂O— and —CH₂CH(CH₃)O—, may be arranged in any desired order, preferably in random, gradient-like or block-like manner, more preferably in block-like manner, and x1 is greater than y1.

Particularly preferred is a process for the preparation of the surface active agents of the formula (Ia1)

R¹—O [symbol] (CH₂CH₂O)ₓ₁ [symbol] —C(O)—CH₂CH₂—C(O)—CH₃ (Ia1)
wherein
a compound of the formula (IIa1)

R¹—O [symbol] (CH₂CH₂O)ₓ₁ [symbol] H (IIa1)
is esterified in presence of at least one acidic esterification catalyst
or transesterified in presence of at least one transesterification catalyst with
a compound of the formula (IIIa1)

R⁵⁰—C(O)—CH₂CH₂—C(O)—CH₃ (Illa1),
wherein in the formulae
R¹ is a linear or branched alkyl group having 16 to 18 carbon atoms or a linear or branched alkenyl group having one or more, preferably 1 to 3, double bonds and 16 to 18 carbon atoms;
x1 is, based on a molar average, a number from 8 to 10;
R⁵ denotes a C₁ to C₄-alkyl group, preferably ethyl or methyl, more preferably methyl, or hydrogen (H).

Compounds (alcohol polyglycol ethers, in particular fatty alcohol polyglcol ethers) of the formula (II), which are considered as starting materials for the production of surface active agents of the formula (I) according to the invention, are known substances which are produced industrially by addition of ethylene oxide and optionally propylene oxide onto the primary alcohol R¹OH, wherein R¹ is as defined above.

Preferably the ethylene oxide used for the preparation of compound (II) is a bio-ethylene oxide obtained from renewable resources. Preferably the optional propylene oxide used for the preparation of compound (II) is a bio-propylene oxide obtained from renewable resources.

Depending on the nature of the catalyst used, the products can have a conventional or a narrowed homologue distribution.

Typical examples of the compound of formula (II) are addition products of 1 to 25 mol, preferably 2 to 20 mol, more preferably 2 to 15 mol, of ethylene oxide onto alcohols R¹—OH as mentioned above. Such alcohols R¹—OH are obtained, for example, in the hydrogenation of technical fatty acid ester fractions or aldehydes from the oxo process.

The compound of the formula (III) is preferably a bio-based ketocarboxylic acid or bio-based ketocarboxylic acid ester, more preferably levulinic acid or levulinic acid C₁ to C₄-alkyl ester (e.g. levulinic acid methyl ester).

As mentioned above the levulinic acid employed is preferably a second-generation bio-based product which stems from cellulosic feedstocks like wheat straw, lignocellulose or sugarcane bagasse.

The esterification of the compound of the formula (II) with the ketocarboxylic acid of formula (III) (R⁵═H) can be carried out in a manner known by a person skilled in the art.

Examples of suitable acidic catalysts are mineral acids, sulfonic acids or acidic ion exchangers. Typical examples are sulfuric acid, methane sulfonic acid, p-toluene sulfonic acid, p-dodecyl benzene sulfonic acid or surfactant sulfonic acids. It is advisable to use the at least one catalyst in amounts of 0.01 to 5% by weight, based on the ketocarboxylic acid of formula (III).

The esterification reaction can be carried out at temperatures from 80 to 230° C., preferably 130 to 190° C. In order to shift the reaction equilibrium to product side, it is advisable to remove the condensation water continuously. This may be facilitated by reducing the pressure in the reaction vessel by applying vacuum. If desired, the acid catalyst can be neutralized after the esterification.

Transesterification of the compounds of formula (II) polyglycol ethers with the ketocarboxylic acid ester of formula (III) (R⁵═C₁-C₄-alkyl, i.e. methyl) can be carried out in a manner known by a person skilled in the art.

Examples of suitable transesterification catalysts are based on alkali or earth alkali metals, lanthanoids, titanium, zirconium, aluminum, zinc, iron, tin or boron. Typical examples are but not limited to alkali and earth alkali hydroxides such as sodium hydroxide, alkali and earth alkali alkoxides such as sodium methoxide, titanium alkoxides such as titanium (IV) tetra isopropoxide or titanium (IV) tetra butoxide, zirconium acetylacetonate, zinc acetate, zinc (II) chloride, zinc (II) oxide, iron (III) chloride, tin oxalate, dibutyl tin methoxide, boron tribromide.

Preferably a Lewis acid based on titanium, zirconium, aluminum, zinc and/or tin is used as transesterification catalyst.

It is advisable to use the catalysts in amounts of 0.01 to 5% by weight, based on the carboxylic acid esters.

The transesterification reaction can also be carried out at temperatures from 80 to 230° C. In order to shift the reaction equilibrium to product side, it is advisable to remove the condensation alcohol continuously. This may be facilitated by reducing the pressure in the reaction vessel by applying vacuum.

According to a preferred embodiment in the process according to the invention as compound (II) a fatty alcohol polyglcol ether is used which is the addition product of bio-ethylene, and optionally bio-propylene, and a natural fatty alcohol R¹OH, and as compound (III) a bio-based ketocarboxylic acid or bio-based ketocarboxylic acid ester, in particular bio-based ketocarboxylic acid methyl ester, and more preferably levulinic acid or levulinic acid ester, in particular levulinic acid methyl ester, is used.

By use of said afore-mentioned bio-based compounds (II) and (III) in the process according to the invention a particularly sustainable, low foaming surfactant agent of formula (I) is provided which has a renewable carbon index of 100%.

The surface active agents of formula (I), in particular formula (Ia), according to the invention have a good foam-suppressing action and good surface activity.

A further aspect of the invention therefore relates to the use of the surface active agents of formula (I) according to the invention for foam regulation and minimization in detergents, automatic dishwashing and laundry detergents, industrial cleaners, and formulations for metal working, textile processing, crop protection, pigment dispersions, and as auxiliaries for the sugar and yeast industry and in the wastewater treatment of sewage treatment plants.

A further aspect of the invention therefore relates to a detergent composition for machine dishwashing comprising one or more the surface active agents of formula (I) according to the invention.

Preferably the detergent composition for machine dishwashing comprises 0.1 to 15 wt.-%, preferably 0.1 to 10 wt.-%, most preferably 0.1 to 5.0 wt.-%, even more preferred 0.5 to 5.0 wt.-%, in particular 1 to 5 wt.-%, of one or more of the surface-active agents of formula (I).

Preferred is a detergent composition comprising:
Z1) one or more of surface-active agents of formula (I), in particular formula (Ia), and further one or more components Z2) to Z7) selected from:
Z2) one or more enzymes, preferably selected from the group consisting of proteases, amylases, lipases, hemicellulases, cellulases, perhydrolases and oxidoreductases;
Z3) one or more builders;
Z4) one or more bleaching agents;
Z5) one or more surfactants
Z6) one or more polymers
Z7) one or more further additives, preferably selected from the group consisting of chelating agents, glass corrosion inhibitors, water, organic solvents, thickeners, foaming inhibitors, color particles, silver protecting agents, agents for preventing the tarnishing of silver, corrosion inhibitors, colorants, fillers, germicidal agents, hydrotropic agents, antioxidants, enzyme stabilizers, perfumes, solubilizers, carriers, processing aids, pigments and pH regulators.

Preferably, the one or more enzymes of component Z2), if present, are selected from the group consisting of proteases, amylases, lipases, hemicellulases, cellulases, perhydrolases and oxidoreductases.

The enzymes are typically of natural origin. Improved variants that are based on natural molecules are obtainable for the use in detergent compositions for machine dishwashing and are accordingly preferred.

Among the proteases, those of the subtilisin type are preferred. Examples are the subtilisins BPN′ and Carlsberg, as well as their advanced forms, protease PB92, subtilisins 147 and 309, alkaline protease from Bacillus lentus, subtilisin DY and subtilases.

Examples for amylases applicable according to the invention are alpha-amylases from Bacillus licheniformis, from B. amyloliquefaciens, from B. stearothermophilus, from Aspergillus niger and A. oryzae as well as their derivatives improved for use in detergent compositions for machine dishwashing. Furthermore, alpha-amylase from Bacillus sp. A 7-7 (DSM 12368) and the cyclodextrin-glucanotransferase (CGTase) FROM B. agaradherens (DSM 9948) are preferred.

Furthermore, lipases or cutinases may be used in the detergent composition of the invention, in particular due to their triglyceride-cleaving activities, but also for in situ preparing peroxy acids from appropriate precursors. Exemplary lipases are those originating from Humicola lanuginosa (Thermomyces lanuginosus) or their advanced forms, especially those with the amino acid exchange D96L. Exemplary cutinases are those originally isolable from Fusarium solani pisi and Humicola insolens.

Moreover, enzymes can be used that are pooled under the term hemicellulases. These are, for example, mannanases, xanthanlyases, pektinlyases (=pektinases), pektinesterases, pektatlyases, xyloglucanases (=xylanases), pullulanases and beta-glucanases.

For increasing the whitening effect, the detergent composition of the invention may also comprise oxidoreductases, for example oxidases, oxygenases, katalases, peroxidases, such as halo-, chloro-, bromo-, lignin-, glucose oder mangan-peroxidases, dioxygenases or laccases (phenoloxidases, polyphenoloxidases). Advantageously, additional, preferably organic, more preferably aromatic compounds that interact with the enzymes, are added in order to increase the activity of the corresponding oxidoreductases (enhancers) or in order to facilitate the electron transport between the oxidizing enzymes and the contaminations if their redox potentials have a large difference (mediators).

The enzymes may be used in any form known in the art, for example as granulated, extruded or lyophilized solid preparations or, particularly in liquid or gel formulations, as solutions of the enzymes, preferably highly concentrated, with a low water content and/or mixed with stabilizers.

Alternatively, the enzymes may be in an encapsulated form, both in solid and in liquid compositions. For example, an enzyme solution may be spray-dried or extruded together with a preferably natural polymer or in the form of capsules, e.g. such, where the enzymes are enclosed in a solidified gel or such of the core-shell type, where an enzyme-containing core is coated with a water-, air- and/or chemical-impermeable protective coating. In additional layers, further active agents such as stabilizers, emulsifiers, pigments, whiteners or dyes may be included. Such capsules are prepared by known methods, e.g. shaking granulation or rolling granulation or in fluidized bed processes. Advantageously, such granulates are coated with polymeric film-forming agents and therefore low on dust and storage stable.

Furthermore, it is possible to compound two or more enzymes to prepare granules with multiple enzymatic activities.

The detergent composition for machine dishwashing according to the invention comprises the one or more enzymes Z2) preferably in amounts of from 1×10⁻⁶ to 5 wt.-%, more preferably from 1×10⁻⁵ to 3 wt.-%, even more preferably from 1×10⁻⁴ to 2 wt.-%, based on the total weight of the detergent composition.

This amount relates to active protein. The protein concentration can be determined by known methods such as the BCA-method or the biuret method.

The builders Z3) as well as other ingredients which may be used in detergent composition of the present invention are disclosed, e.g. in US 2010/0160204 and EP-A 1757676.

The builders Z3), if present, may be selected, e.g., from the group consisting of carbonates, bicarbonates, organic builders, preferably methylglycinediacetic acid (MGDA), silicates, phosphates, phosphonates and alkali metal hydroxides.

Preference is given to the use of carbonate(s) and/or bicarbonate(s), preferably alkali metal carbonate(s), more preferably sodium carbonate.

These substances are preferably used in amounts of from 2 to 50 wt.-%, preferably from 20 to 40 wt.-% and in particular from 25 to 35 wt.-%, based on the total weight of the detergent composition according to the invention.

Organic builders include polycarboxylates, polycarboxylic acids, polymeric carboxylates, aspartic acid, polyacetals, and dextrins. Useful organic builders are, among others, polycarboxylic acids usable in the form of the free acid and/or their sodium salts, wherein polycarboxylic acids are understood as those carboxylic acids, which carry more than one acid moiety. For example, these may be citric acid, adipic acid, succinic acid, glutaric acid, malic acid, tartaric acid, maleic acid, fumaric acid, sugar acids, aminocarboxylic acids and nitrilotriacetic acid (NTA) and mixtures thereof. In addition to their builder effect, the free acids typically also have the property of an acidifying agent and thus, also serve to set a lower and milder pH for the detergent composition according to the invention. In particular, citric acid, succinic acid, glutaric acid, adipic acid, gluconic acid, tartaric acid and any mixtures thereof are suitable.

Common aminocarboxylic acids that are preferred in the context of the present invention are, for example, ethylenediaminetetraacetic acid (EDTA), methylglycine-diacetic acid (MGDA) and glutamic diacetic acid (GLDA) or mixtures thereof.

Further preferred builders are polymeric aminodicarboxylic acids, their salts or their precursors. Particular preference is given to using polyaspartic acids, their salts or their derivatives.

Particularly advantageous for the cleaning and rinse aiding properties of the detergent composition of the invention is the use of citric acid and/or citrates. Preference is given to detergent compositions which contain citric acid or a salt of citric acid, the weight fraction of citric acid or of the salt of citric acid preferably being from 2 to 50 wt.-%, more preferably from 5 to 30 wt.-% and even more preferably from 10 to 30 wt.-%, based on the total weight of the detergent composition.

In another preferred embodiment of the invention, the automatic dishwashing compositions according to the invention contain MGDA as one of their builders. Machine dishwashing detergents according to the invention preferably contain from 0.5 to 25 wt.-%, more preferably from 2 to 25 wt.-% of MGDA, based on the total weight of the detergent composition.

As organic builders, polymeric carboxylates are also suitable. These are, for example, the alkali metal salts of polyacrylic acid or polymethacrylic acid, for example, those having a molecular weight of 500 to 70,000 g/mol. Suitable polymeric carboxylates are in particular polyacrylates which preferably have a molecular weight of 2,000 to 20,000 g/mol. Because of their superior solubility, the short-chain polyacrylates which have molar masses of from 2000 to 10 000 g/mol and more preferably from 3000 to 5000 g/mol are even more preferred in this group.

Also suitable are copolymeric carboxylates. Suitable comonomers are monoethylenically unsaturated dicarboxylic acids such as maleic acid, fumaric acid, maleic anhydride, itaconic acid and citraconic acid. Particularly suitable are copolymeric carboxylates of acrylic acid with methacrylic acid and of acrylic acid or methacrylic acid with maleic acid and/or fumaric acid. Copolymers of acrylic acid with maleic acid which contain 50 to 90 wt.-% of acrylic acid and 10 to 50 wt.-% of maleic acid have proven to be particularly suitable. Their molecular weight relative to free acids is preferably from 2000 to 70,000 g/mol, more preferably from 20,000 to 50,000 g/mol and in particular from 30,000 to 40,000 g/mol. It is also possible to use copolymers of at least one monomer selected from the group consisting of monoethylenically unsaturated C₃-C₁₀ mono- or C₄-C₁₀-dicarboxylic acids or their anhydrides, such as maleic acid, maleic anhydride, acrylic acid, methacrylic acid, fumaric acid, itaconic acid and citraconic acid with at least one hydrophilic or hydrophobic modified monomer, as listed below, are used.

Suitable hydrophobic monomers are, for example, isobutene, diisobutene, butane, pentene, hexene and styrene, olefins having 10 or more carbon atoms or mixtures thereof, for example 1-decene, 1-dodecene, 1-tetradecene, 1-hexadecene, 1-octadecene, 1-eicosene, 1-docosene, 1-tetracosene and 1-hexacosene, C₂₂-α-olefin, a mixture of C₂₀-C₂₄-α-olefins and polyisobutene having a number-average of 12 to 100 carbon atoms per molecule.

Suitable hydrophilic monomers are monomers with sulfonate or phosphonate groups and nonionic monomers with hydroxyl function or alkylene oxide groups and optionally other ionogenic or nonionogenic monomers. Examples of the above listed hydrophilic monomers are allyl alcohol, isoprenol, methoxypolyethylene glycol(meth)acrylate, methoxypolypropylene glycol (meth)acrylate, methoxypolybutylene glycol (meth)acrylate, methoxypoly(propylene oxide-co-ethylene oxide) (meth)acrylate, ethoxypolyethylene glycol (meth)acrylate, ethoxypolypropylene glycol (meth)acrylate, ethoxypolybutylene glycol (meth)acrylate and ethoxypoly(propylene oxide-co-ethylene oxide) (meth)acrylate. Polyalkylene glycols may contain 3 to 50, in particular 5 to 40 and especially 10 to 30 alkylene oxide units per molecule.

Particularly preferred monomers containing sulfonic acid groups are 1-acrylamido-1-propanesulfonic acid, 2-acrylamido-2-propanesulfonic acid, 2-acrylamido-2-methylpropanesulfonic acid (2-acryloylamino-2-methylpropane-sulfonic acid), 2-methacrylamido-2-methylpropanesulfonic acid, 3-methacrylamido-2-hydroxypropanesulfonic acid, allylsulfonic acid, methallylsulfonic acid, allyloxybenzenesulfonic acid, methallyloxybenzenesulfonic acid, 2-hydroxy-3-(2-propenyloxy) propanesulfonic acid, 2-methyl-2-propene-1-sulfonic acid, styrenesulfonic acid, vinylsulfonic acid, 3-sulfopropyl acrylate, 2-sulfoethyl methacrylate, 3-sulfopropylmethacrylat, sulfomethacrylamide, sulfomethylmethacrylamide and salts of said acids, such as their sodium, potassium or ammonium salts.

Particularly preferred phosphonate group-containing monomers are vinylphosphonic acid and its salts. Moreover, amphoteric polymers can also be used as builders.

When the detergent composition according to the invention comprises one or more (co) polymeric carboxylates, the amount of these (co) polymeric carboxylates in the automatic dishwashing agent according to the invention is preferably 0.5 to 20 wt.-% by weight and in particular 3 to 10 wt.-%, based on the total weight of the detergent composition of the invention.

Oxidisuccinates and other derivatives of disuccinates, preferably ethylenediamine disuccinate are further preferred organic builders, often referred to as co-builders. Among these, ethylenediamine-N,N′-disuccinate (EDDS) is preferred, especially used in the form of its sodium or magnesium salts. Furthermore as a builder or co-builder preferred in this context are glycerol disuccinates and glycerol trisuccinates.

The detergent composition according to the invention may preferably comprise as builders crystalline sodium sheet silicates of the general formula NaMSiₓO₂ₓ₊₁·yH₂O, wherein M is sodium or hydrogen, x is a number from 1.9 to 22, preferably from 1.9 to 4, more preferably 2, 3 or 4, and y is a number from 0 to 33, preferably from 0 to 20.

The detergent composition according to the invention preferably contains the crystalline sodium sheet silicates of formula NaMSiₓO₂ₓ₊₁·yH₂O in an amount of 0.1 to 20 wt.-%, more preferably from 0.2 to 15 wt.-%, even more preferably from 0.4 to 10 wt.-%, based on the total weight of the detergent composition for machine dishwashing.

It is also possible to use amorphous sodium silicates having a modulus Na₂O:SiO₂ of from 1:2 to 1:3.3, preferably from 1:2 to 1:2.8 and in particular from 1:2 to 1:2.6, which preferably show delayed dissolution and secondary wash properties. The dissolution delay compared to conventional amorphous sodium silicates can be caused in different ways, for example by surface treatment, compounding, compaction, condensing or over-drying. In the context of this invention, the term “amorphous” means that the silicates in do not produce sharp X-ray reflections typical of crystalline substances in X-ray diffraction experiments, but at most cause one or more maxima of the scattered X-ray radiation, which have a width of multiple degrees of the diffraction angle.

Alternatively or in combination with the aforementioned amorphous sodium silicates X-ray-amorphous silicates can be used, the silicate particles of which show blurred or even sharp diffraction maxima in Electron diffraction experiments.

This is to be interpreted as meaning that the products have microcrystalline regions of the size of ten to a few hundred nm, with values of up to a maximum of 50 nm and in particular up to a maximum of 20 nm being preferred. Such X-ray amorphous silicates also have a dissolution delay compared to the conventional water glasses. Particularly preferred are condensed/compacted amorphous silicates, compounded amorphous silicates and overdried X-ray-amorphous silicates. In the context of the present invention, it is preferred that these silicates, preferably alkali metal silicates, particularly preferably crystalline or amorphous alkalidisilicates, are present in the detergent composition for machine dishwashing of the invention in amounts of from 3 to 60 wt.-%, preferably from 8 to 50 wt.-% and more preferably from 20 to 40 wt.-%, based on the total weight of the detergent composition.

Phosphates have proven to be effective builders in terms of cleaning performance. Among the large number of commercially available phosphates, the alkali metal phosphates have the greatest importance in the washing and cleaning industry, in particular pentasodium triphosphate or pentapotassium triphosphate (sodium or potassium tripolyphosphate).

Alkali metal phosphates is the summary term for the alkali metal salts (especially sodium and potassium salts) of the various phosphoric acids such as metaphosphoric acids (HPO₃)ₘ, orthophosphoric acid H₃PO₄ and higher molecular weight representatives. The phosphates combine several advantages: they act as alkali metal carriers, prevent lime deposits on machine parts and contribute to the cleaning performance of the detergent composition.

Technically particularly important phosphates are the pentasodium triphosphate NasP₃O₁₀ (sodium tripolyphosphate) and the corresponding potassium salt pentapotassium triphosphate K₅P₃O₁₀ (potassium tripolyphosphate). The sodium potassium tripolyphosphates are also preferably used according to the invention. If phosphates are used in the detergent composition according to the invention, preferred agents comprise phosphate(s), preferably alkali metal phosphate(s), more preferably pentasodium or pentapotassiumtriphosphat (sodium or potassium tripolyphosphate), in amounts of from 2 to 50 wt.-%, preferably from 2 to 30 wt.-%, more preferably from 3 to 25 wt.-% and particularly preferably from 3 to 15 wt.-%, based on the total weight of the detergent composition according to the invention.

As further builders, the detergent composition for machine dishwashing according to the invention may contain one or more phosphonates, which are often referred to as co-builders. The amount of phosphonates in the detergent composition of the invention is preferably 0.5 to 20 wt.-% and more preferably 1.0 to 10 wt.-%, based on the total weight of the detergent composition.

The chelating phosphonates include a number of different compounds such as 1-hydroxyethane-1,1-diphosphonic acid (HEDP) or diethylenetriamine penta(methylenephosphonic acid) (DTPMP). Particularly preferred are hydroxyalkane and aminoalkane phosphonates. Among the hydroxyalkane phosphonates, 1-hydroxyethane-1,1-diphosphonate (HEDP) is of particular importance, preferably as a co-builder. It is preferably used as a sodium salt, of which the disodium salt reacts neutral and the tetrasodium salt reacts alkaline (pH 9). As aminoalkanphosphonates, ethylenediamine tetramethylene phosphonate (EDTMP), diethylenetriamine pentamethylenephosphonate (DTPMP) and their higher homologues, among others, are applicable. They are preferably used in the form of neutral reacting sodium salts (e.g. as the hexasodium salt of EDTMP or as hepta- and octasodium salt of DTPMP). From the class of phosphonates, HEDP is preferably used.

Detergent compositions according to the invention can contain, as further builders, alkali metal hydroxides. These alkali carriers are preferably only used in small amounts, typically in amounts of 10 wt.-% or less, preferably 6 wt.-% or less, more preferably 5 wt.-% or less, even more preferably 0.1 to 5 wt. % and in particular 0.5 to 5 wt.-%, based on the total weight of the detergent composition.

In a further preferred embodiment of the invention, the detergent composition according to the invention comprises one or more builders from the group of organic builders. In a particularly preferred embodiment of the invention, the detergent composition contains one or more builders from the group consisting of citrate, methylglycinediacetic acid (MGDA) and ethylenediamine-N,N′-disuccinate (EDDS). In a particularly preferred embodiment of the invention, the automatic dishwasher detergents according to the invention contain MGDA, specifically its trisodium salt.

The detergent composition according to the invention can contain said builders both individually and in the form of mixtures of two, three, four or more builders. In a preferred embodiment of the invention, the detergent composition according to the invention does not contain any phosphate builders.

The detergent composition according to the invention contains the one or more builders of component Z3) preferably in amounts of from 2 to 80 wt.-%, more preferably in amounts of from 30 to 80 wt.-%, especially preferably in amounts of from 40 to 75 wt.-%, and most preferably in amounts of from 50 to 75 wt.-%, based on the total weight of the detergent composition for machine dishwashing according to the invention.

The bleaching agent Z4) of the detergent composition for machine dishwashing according to the invention, if present, preferably contains one or more substances selected from the group consisting of bleaches, bleach activators and bleach catalysts.

As bleach, the detergent composition of the invention may contain an oxygen bleach. Among these oxygen bleaches, which yield H₂O₂ in water, sodium percarbonate, sodium perborate tetrahydrate and sodium perborate monohydrate are of particular importance. Further applicable bleaches are, for example, peroxypyrophosphates, citrate perhydrates and H₂O₂-yielding peroxy acid salts or peroxy acids, such as peroxy benzoates, peroxy phthalates, diperoxy azelaic acid, Phthaloiminoperoxy acid or diperoxy dodecanedioic acid. Organic bleaches can also be used. Typical organic bleaches are diacyl peroxides, such as dibenzoyl peroxide. Other typical organic bleaches are the peroxy acids, such as alkyl peroxy acids and aryl peroxy acids.

The detergent composition according to the invention preferably comprises one or more bleaches selected from the group consisting of oxygen bleaches, peroxy pyrophosphates, citrate perhydrates, and H₂O₂-delivering peroxy acid salts or peroxy acids and organic bleaches. Particularly preferably, the detergent composition contains 1.0 to 20 wt.-%, preferably 4.0 to 18 wt.-% and more preferably 8 to 15 wt.-% of an oxygen bleach, preferably sodium percarbonate, based on the total weight of the detergent composition for machine dishwashing.

To achieve an improved bleaching effect when dishwashing at temperatures of about 60° C. and below, the inventive detergent composition may additionally contain one or more bleach activators. Preferably, the one or more bleach activators are selected from the group consisting of which, under the conditions of perhydrolysis, result in aliphatic peroxycarboxylic acids having preferably 1 to 10 carbon atoms, in particular 2 to 4 carbon atoms, and/or optionally substituted perbenzoic acid. Suitable substances are those which carry O- and/or N-acyl groups with the above-stated number of carbon atoms and/or optionally substituted benzoyl groups. Preference is given to polyacylated alkylenediamines, with tetraacetylethylenediamine (TAED) being particularly suitable.

Bleach activators, in particular TAED, are preferably used in amounts of up to 10 wt.-% by weight, more preferably in amounts of from 0.1 to 8 wt.-% even more preferably in amounts of from 2 to 8 wt.-%, particularly in amounts from 2 to 6 wt.-%, based on the total weight of the detergent composition according to the invention.

In addition to, or in place of, the conventional bleach activators, so-called bleach catalysts can also be used. These substances are bleach-enhancing transition metal salts or transition metal complexes such as Mn, Fe, Co, Ru or Mo-salen complexes or carbonyl complexes. Mn, Fe, Co, Ru, Mo, Ti, V and Cu complexes with nitrogen-containing tripod ligands and Co, Fe, Cu and Ru amine complexes can also be used as bleach catalysts.

It is particularly preferred to use complexes of manganese in the oxidation state II, III, IV or V, which preferably contain one or more macrocyclic ligands with donor functions N, NR, PR, O and/or S. Preferred are ligands having nitrogen donor functions. It is particularly preferred to use bleach catalyst(s) which contain as macromolecular ligands 1,4,7-trimethyl-1,4,7-triazacyclononane (Me-TACN), 1,4,7-triazacyclononane (TACN), 1,5,9-trimethyl-1,5,9-triazacyclododecane (Me-TACD), 2-methyl-1,4,7-trimethyl-1,4,7-triazacyclononane (Me/Me-TACN) and/or 2-methyl-1,4,7-triazacyclononane (Me/TACN). Suitable manganese complexes are, for example, [Mn^{III}₂(μ-O)₁(μ-OAc)₂(TACN)₂](ClO₄) 2, [Mn^{III}Mn^{IV}(μ-O)₂(μ-OAc)₁(TACN)₂](BPh₄)₂, [Mn^{III}₄(μ-O)₆(TACN)₄](ClO₄)₄, [Mn^{III}₂(μ-O)₁(μ-OAc)₂(Me-TACN)₂](ClO₄)₂, [Mn^{III}Mn^{IV}(μ-O)₁(μ-OAc)₂(Me-TACN)₂](ClO₄)₃, [Mn^{IV}₂(μ-O)₃(Me-TACN)₂](PF₆)₂ and [Mn^{IV}₂ (μ-O)₃(Me/Me-TACN)₂](PF₆)₂(OAc═OC(O)CH₃).

In a further preferred embodiment of the invention, the detergent composition according to the invention comprises one or more bleach catalysts from the group of bleach-enhancing transition metal salts and transition metal complexes, preferably from the group of the complexes of manganese with 1,4,7-trimethyl-1,4,7-triazacyclononane (Me-TACN) and 1,2,4,7-tetramethyl-1,4,7-triazacyclononane (Me₄-TACN), since the cleaning result can be significantly improved by these bleach catalysts.

Preferably, the bleaching agent Z4) comprises one or more bleaches and one or more substances from the group of bleach activators and bleach catalysts. More preferably, the bleaching agent comprises one or more bleaches, one or more bleach activators, and one or more bleach catalysts.

The detergent composition according to the invention contains the bleaching agent of component Z4) preferably in amounts of 1 to 40 wt.-%, more preferably in amounts of 0.5 to 30 wt.-% and even more preferably in amounts of 3 to 25 wt.-%, based on the total weight of the detergent composition.

The one or more surfactants of component Z5) of the detergent composition according to the invention, if present, are different from Z1) and preferably selected from the group consisting of nonionic surfactants, zwitterionic surfactants, anionic surfactants, cationic surfactants and mixtures thereof.

The one or more nonionic surfactants of component Z5) of the detergent composition according to the invention preferably has a cloud point of 40 to 60° C.

The detergent composition according to the invention contains the one or more nonionic surfactants of component Z5) preferably in amounts of from 0 to 15 wt.-%, more preferably in amounts of from 0.1 to 10 wt.-% and even more preferably in amounts of 0.2 to 5 wt.-%, each based on the total weight of the detergent composition according to the invention.

Preference is also given to detergent compositions according to the invention, where the one or more surfactants of component Z5) are selected from the group of one or more modified fatty alcohol alkoxylates of the formula (IV)

RₐO-(AO)ₓ′—Y (IV),
in which
Rₐ is a linear or branched saturated alkyl group having 8 to 30 carbon atoms, or a linear or branched unsaturated alkenyl group having one or more double bonds and 8 to 30 carbon atoms,
A is selected from the group consisting of —C₂H₄— and —C₃H₆—,
x′ is a number from 1 to 150,
Y is a group —CH₂—CH(OH)—R_{b} or is a linear or branched saturated alkyl group having 1 to 30 carbon atoms, preferably-CH₂—CH(OH)—R_{b},
   R_{b} is a linear or branched saturated alkyl group having 1 to 30 carbon atoms, and where the group -(AO)_{x′}— comprises one or more-C₂H₄—O— groups and may additionally comprise one or more-C₃H₆—O— groups, and, when the group -(AO)_{x′}— simultaneously comprises-C₂H₄—O— and —C₃H₆—O— groups, the —C₂H₄—O— and —C₃H₆—O— groups may be distributed over the
   -(AO)_{x′}— group in any desired way, preferably in random, gradient-like or block-like manner and more preferably in block-like manner, and the molar amount of the —C₂H₄—O— groups in the group -(AO)_{x′}— is preferably greater than the molar amount of the —C₃H₆—O— groups in the group -(AO)_{x′}—.

Preference is also given to detergent compositions according to the invention, where the one or more surfactants of component Z5) are selected from the group of one or more fatty alcohol alkoxylates of the formula (V)

RₐO-(AO)_{x′}—H (V).
in which
Rₐ is a linear or branched saturated alkyl group having 8 to 30 carbon atoms, or a linear or branched unsaturated alkenyl group having one or more double bonds and 8 to 30 carbon atoms,
A is selected from the group consisting of —C₂H₄— and —C₃H₆—,
x′ is a number from 1 to 150.

Examples for the alkyl- and alkenyl-groups of Rₐ of formulae (IV) and (V) of component Z5) are, e.g. the alkyl and alkenyl groups of the following alcohols Rₐ—OH: 1-octanol (caprylic alcohol), 2-ethylhexanol, 1-nonanol (pelargonic alcohol), 1-decanol (capric alcohol), 1-undecanol, 1-dodecanol (lauryl alcohol), 1-tridecanol, isotridecanol, 1-tetradecanol (myristyl alcohol), 1-pentadecanol, 1-hexadecanol (cetyl alcohol), cis-9-hexadecene-1-ol (palmitoleyl alcohol), 1-heptadecanol, 1-octadecanol (stearyl alcohol), cetearyl alcohol, 16-methylheptadecan-1-ol (Isostearyl alcohol), 9E-octadecene-1-ol (elaidyl alcohol), cis-9-octadecene-1-ol (oleyl alcohol), oleyl cetyl alcohol (i.e., a mixture of oleyl alcohol and cetyl alcohol), 9Z,12Z-octadecadien-1-ol (linoleyl alcohol), 9E,12E-octadecadien-1-ol (Elaidolinoleyl alcohol), 9Z,12Z,15Z-octadecatrien-1-ol (linolenyl alcohol), 9E,12E,15E-octadecatriene-1-ol (elaidolinolenyl alcohol), 1-nonadecanol, 1-eicosanol (arachidyl alcohol), 1-heneicosanol, 1-docosanol (behenyl alcohol), cis-13-docosen-1-ol (erucyl alcohol), 1-tetracosanol (lignoceryl alcohol), 1-hexacosanol (ceryl alcohol), 1-octacosanol (montanyl alcohol) and 1-triacontanol (myricyl alcohol) or mixtures thereof.

Rₐ in formulae (IV) and (V) preferably represents a linear or branched saturated alkyl group having 8 to 22 carbon atoms or a linear or branched unsaturated alkenyl group having one or more double bonds and 8 to 22 carbon atoms, more preferably a linear or branched saturated alkyl group having 8 to 18 carbon atoms or a linear or branched unsaturated alkenyl group having one or more double bonds and 8 to 18 carbon atoms, and particularly preferably a linear or branched saturated alkyl group having 1 to 15 carbon atoms or a linear or branched unsaturated alkenyl group having one or more double bonds and 10 to 15 carbon atoms.

Preferably, the groups Rₐ in formulae (IV) and (V) are alkyl groups.

Preferably, z in formulae (IV) and (V) is a number from 1 to 50, more preferably from 1 to 20, and even more preferably from 5 to 20.

The variable x′ in the one or more compounds of formulae (IV) and (V) represents molar averages, i.e. the detergent composition according to the invention may contain several compounds of formulae (IV) and (V) with different degrees of alkoxylation.

In a further preferred embodiment of the invention, the group -(AO)_{x′}— of formulae (IV) and (V) contains one or more-C₂H₄O groups and one or more —C₃H₆O groups. In this preferred embodiment of the invention, the molar amount of the —C₃H₆O groups, based on the total amount of —C₂H₄O and C₃H₆O groups, is preferably less than 50%, more preferably 45% or less than 45%, even more preferably 40% or less than 40%, and particularly preferably 33% or less than 33%.

In a preferred embodiment of the invention, the molar amount of —C₃H₆O groups, based on the total amount of —C₂H₄O and —C₃H₆O groups, is preferably 20 to less than 50%, more preferably 33 to 45%, and even more preferably 33 to 40%.

The molar amount of the —CH₆O groups, based on the total amount of —C₂H₄O and —C₃H₆O groups, is preferably 20% or less than 20% and particularly preferably 10% or less than 10%.

In a particularly preferred embodiment of the invention, the surfactant of formula (V) contains a molar average of 8 —C₂H₄O groups and 4 —C₃H₆O groups and Rₐ represents a linear or branched saturated alkyl group having 12 to 15 carbon atoms or a linear or branched unsaturated alkenyl group having one or more double bonds and 12 to 15 carbon atoms.

Preference is also given to detergent compositions according to the invention, where the one or more surfactants of component Z5) are selected from the group of N-acylglucamines, which are also known as N-1-deoxysorbityl fatty acid amides or glucamides, of the formula (VI)

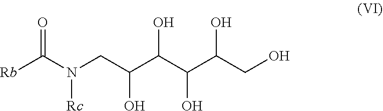

wherein
Rb is a linear or branched saturated alkyl group having 11 to 21 Carbon atoms or a linear or branched unsaturated alkenyl group having one or more double bonds and 11 to 21 carbon atoms and
Rc is hydrogen or a linear or branched saturated alkyl group having 1 to 4 carbon atoms.

Preferably, in the one or more N-acylglucamines of formula (VI), Rc is a methyl group.

In the one or more N-acylglucamines of the formula (VI), Rb is preferably a linear or branched saturated alkyl group having 11 to 17 carbon atoms or a linear or branched unsaturated alkenyl group with one or more double bonds and 11 to 17 carbon atoms.

More preferably, in the one or more N-acylglucamines of formula (VI) Rb is a linear or branched saturated alkyl group having 15 to 17 carbon atoms or a linear or branched unsaturated alkenyl group having one or more double bonds and 15 to 17 carbon atoms.

In a preferred embodiment of the invention, 50 wt.-% or more, more preferably 60 to 99 wt.-% and even more preferably 70 to 98 wt.-% of the groups Rb in the one or more N-acylglucamines of the formula (VI) are linear or branched saturated alkyl groups with 17 carbon atoms.

In a further preferred embodiment of the invention, 0.1 to 50 wt.-%, more preferably 0.5 to 40 wt.-% and even more preferably 1.0 to 30 wt.-% of the groups Rb in the one or more N-acylglucamines of the formula (VI) are linear or branched saturated alkyl groups with 15 carbon atoms.

In a further preferred embodiment of the invention, 50 wt.-% or more of the Rb groups in the one or more N-acylglucamines of the formula (VI) are linear or branched unsaturated alkenyl groups having one or more double bonds.

In a particularly preferred embodiment of the invention 50 wt.-% or more, more preferably 80 wt.-% or more and even more preferably 90 wt.-% or more of the groups Rb in the one or more N-acylglucamines of the formula (VI) are linear or branched alkenyl groups having one or more double bonds and 17 carbon atoms.

Particularly preferably, in the one or more N-acylglucamines of formula (VI), Rb is a linear group.

In another preferred embodiment of the invention, in the one or more N-acylglucamines of formula (VI) RbCO derives from lauric acid, palmitic acid, stearic acid, oleic acid, linoleic acid or linolenic acid. In a more preferred embodiment of the invention, in the one or more N-acylglucamines of the formula (VI) RbCO derives from stearic acid, oleic acid, linoleic acid or linolenic acid. In an even more preferred embodiment of the invention, in the one or more N-acylglucamines of formula (VI), RbCO derives from oleic acid, linoleic acid or linolenic acid, and in a particularly preferred embodiment of the invention, in the one or more N-acylglucamines of formula (VI) RbCO derives from oleic acid.

The detergent compositions according to the invention comprise the one or more surfactants of component Z5), preferably in amounts of from 0.1 to 15 wt.-%, more preferably in amounts of from 0.2 to 10 wt.-% even more preferably in amounts of from 0.2 to 5 wt.-%, based on the total weight of the detergent composition for machine dishwashing according to the invention.

Suitable cationic surfactants of component Z5), which may be used instead of or together with the other surfactants mentioned herein, are, e.g., surfactants of formulae (VII), (VIII) and/or (IX)

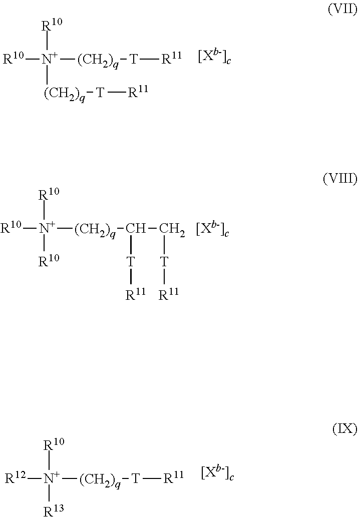

wherein
each R¹⁰ group is independently selected from linear or branched, preferably linear saturated alkyl groups having 1 to 6 carbon atoms, linear or branched, preferably linear, unsaturated alkenyl groups having one or more double bonds and 2 to 6 carbon atoms, and linear or branched, preferably linear hydroxyalkyl groups having 1 to 6 carbon atoms;
each R¹¹ group is independently selected from linear or branched saturated alkyl groups having 8 to 28 carbon atoms, linear or branched unsaturated alkenyl groups having one or more double bonds and 8 to 28 carbon atoms;
R¹² is the same as R¹⁰ or (CH₂) n-T-R¹¹;
R¹³ is the same as R¹⁰, R¹¹ or (CH₂) n-T-R¹¹;
T is selected from —CH₂—, —O—CO— or —CO—O—;
q is an integer from 0 to 5;
X is an inorganic or organic anion having the charge b;
b is a number from 1 to 4; and
c is a number having a value of 1/b.

Further cationic surfactants suitable as component Z5) of the detergent composition according to the present invention are compounds of formula (X),

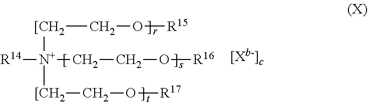

wherein
R¹⁴ is a linear or branched, preferably linear saturated alkyl group having from 1 to 6 carbon atoms;
R¹⁵, R¹⁶ and R¹⁷ are equal or different and are independently selected from the group consisting of hydrogen, linear or branched saturated alkyl groups having from 1 to 18 carbon atoms, linear or branched unsaturated alkenyl groups having one or more double bonds and from 2 to 18 carbon atoms, and —CO—R¹⁸;
R¹⁸ is a linear or branched saturated alkyl group having 7 to 19 carbon atoms or a linear or branched unsaturated alkenyl group having one or more double bonds and 7 to 19 carbon atoms;
r, s and t are equal or different and are independently a number from 0 to 50;
X is an inorganic or organic anion having the charge b;
b is a number from 1 to 4; and
c is a number having a value of 1/b;

with the proviso that at least one group of R¹⁵, R¹⁶ and R¹⁷ is-CO—R¹⁸ which forms an ester moiety with the oxygen atom of an ethoxy group;
the sum of r+s+t is a number from 1 to 70; and
if one or more of r, s and/or t is 0, the corresponding group R¹⁵, R¹⁶ and/or R¹⁷ is a linear or branched saturated alkyl group having 1 to 18 carbon atoms or a linear or branched unsaturated alkenyl group having 2 to 18 carbon atoms.

Preferable zwitterionic surfactants applicable as component Z5) are selected from the group consisting of C₈ to C₁₈, preferably C₁₂ to C₁₈ amine oxides and sulfo- and hydroxyl betaines, such as N-alkyl-N, N-dimethylamino-1-propanesulfonate, wherein the alkyl group may be C₉ to C₁₈, preferably C₁₀ to C₁₄.

Preferable anionic surfactants applicable as component Z5) are selected from alkyl ethoxysulfates having a degree of ethoxylation of more than 3, more preferably 4 to 10 and even more preferably 6 to 8 and an alkyl chain length in the range of C₈ to C₁₆ and preferably C₁₁ to C₁₅. In addition, branched alkyl carboxylates have been found to be useful for the purposes of the present invention when the branching occurs in the middle and the average total chain length is 10 to 18, preferably 12 to 16 with a side chain length of 2 to 4 carbon atoms. An example of this is 2-butyloctanoic acid. The anionic surfactant is usually of a type having good solubility in the presence of calcium. Furthermore, alkyl (polyethoxy) sulfates (AES), alkylbenzene sulfonates and short-chain C₆-C₁₀-alkyl sulfates and sulfonates are among such anionic surfactants. It has been revealed that straight-chain fatty acids are ineffective because of their sensitivity to calcium.

In preferred embodiments, the detergent composition for machine dishwashing according to the present invention comprises cationic and/or zwitterionic surfactants in component Z5) in amounts smaller than 6 wt.-%, preferably smaller than 4 wt.-%, more preferably smaller than 2 wt.-%, even more preferably smaller than 1 wt.-%.

Suitable polymers of component Z6) include washing or cleaning-active polymers, for example rinse aid polymers and/or polymers which act as softeners. In general the detergent composition for machine dishwashing according to the present invention may include, as polymers of component Z6), nonionic, cationic, anionic and/or amphoteric polymers.

Cationic polymers in the context of the present invention are polymers which carry a positive charge in the polymer molecule. This can be realized, for example, by (alkyl) ammonium groups or other positively charged groups present in the polymer chain. Particularly preferred cationic polymers come from the groups of quaternized cellulose derivatives, polysiloxanes with quaternary groups, cationic guar derivatives, polymeric dimethyldiallylammonium salts and their copolymers with esters and amides of acrylic acid and methacrylic acid, copolymers of vinylpyrrolidone with quaternized derivatives of dialkylaminoacrylates and methacrylates, vinylpyrrolidone-methoimidazolinium chloride copolymers, quaternized polyvinyl alcohols, or polymers having the INCI names polyquaternium 2, polyquaternium 17, polyquaternium 18 and polyquaternium 27.

If cationic polymers are used in component Z6), they are particularly preferably copolymers comprising polyalkylene oxide groups and quaternary nitrogen atoms. More preferably the cationic polymers of component Z6) are copolymers comprising
0.1 to 99.9 mol-%, preferably 20.0 to 80.0 mol-%, more preferably 22.0 to 77.6 mol-% of one or more cationic structural units (D); and
0.1 to 99.9 mol-%, preferably 0.4 to 20.0 mol-%, more preferably 0.5 to 4.4 mol-% of one or more macromonomeric structural units (E),
wherein the one or more cationic structural units (D) are represented by the following general formulae (XI) and/or (XII):

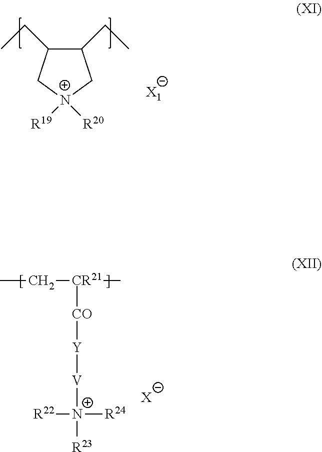

wherein
R¹⁹ and R²¹ are equal or different and are independently selected from hydrogen and/or a methyl group;
R²⁰, R²², R²³ and R²⁴ are equal or different and are independently selected from the group consisting of hydrogen, an aliphatic hydrocarbon residue having 1 to 20, preferably 1 to 4 carbon atoms, a cycloaliphatic hydrocarbon residue having 5 to 20, preferably 5 to 8 carbon atoms, an aryl group having 6 to 14 carbon atoms and/or polyethylene glycol (PEG), and preferably are equal or different and independently selected from the group consisting of hydrogen and/or methyl, and particularly preferably are methyl;
Y is the same or different and is selected from oxygen, NH and/or NR²²,
V is the same or different and is selected from —(CH₂)_{x″}—,

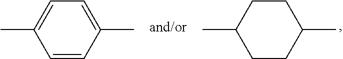

x″ is the same or different and is a number from 1 to 6;
X and X₁ are equal or different and are independently selected from a halogen atom, C₁ to C₄-alkylsulfate and/or C₁ to C₄ alkylsulfonate; and
the one or more macromonomeric structural units (E) are represented by the following general formula (XIII):

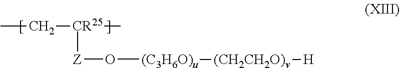

wherein
R²⁵ is the same or different and is H and/or methyl;
Z is the same or different and is C═O and/or O(CH₂)₄, preferably O(CH₂)₄,
u is, on molar average, a number from 0 to 7, preferably from 0 to 6; and
v is, on molar average, a number from 1 to 150, preferably from 11 to 150, more preferably from 12 to 150.

Amphoteric polymers in the context of the present invention have, alongside positively charged groups, also negatively charged groups or monomeric units in the polymer chain. These negatively charged groups or monomeric units may be derived, e.g. from carboxylic acids, sulfonic acids to phosphonic acids.

Preferable amphoteric polymers applicable in component Z6) of the detergent composition of the invention are selected from the group consisting of alkylacrylamide/acrylic acid-copolymers, alkylacrylamide/methacrylic acid copolymers, alkylacrylamide/methylmethacrylic acid copolymers, alkylacrylamide/acrylic acid/alkyl aminoalkyl (meth)acrylic acid copolymers, alkylacrylamide/methacrylic acid/alkylaminoalkyl (meth)acrylic acid copolymers, alkylacrylamide/methylmethacrylic acid/alkylaminoalkyl (meth)acrylic acid copolymers, alkylacrylamide/alkylmethacrylate/alkylaminoethylmethacrylate/alkylmethacrylate copolymers, and copolymers of unsaturated carboxylic acids, cationically derivatized unsaturated carboxylic acids and optionally further ionic or not-ionogenic monomers. Further preferably applicable amphoteric polymers are selected from the group consisting of acrylamidoalkyl-trialkylammonium chloride/acrylic acid copolymers and their alkaline metal or ammonium salts, acrylamidoalkyl-trialkylammonium chloride/methacrylic acid copolymers and their alkaline metal or ammonium salts, and methacroylethylmetaine/methacrylate copolymers.

If the detergent composition for machine dishwashing according to the invention contains cationic and/or amphoteric polymers in component Z6), these polymers are preferably present in amounts of from 0.01 to 10 wt.-%, based on the total weight of the detergent composition. In the context of the present invention, preference is given to those detergent compositions, in which the weight fraction of the cationic and/or amphoteric polymers is from 0.01 to 8 wt.-%, preferably 0.01 to 4 wt.-%, more preferably from 0.01 to 2 wt.-%, even more preferably 0.01 to 1 wt.-%, based on the total weight of the detergent composition.

Another group of preferably applicable polymers applicable in component Z6) of the detergent composition for machine dishwashing of the invention are alkoxylated polyalkyleneimines. Alkoxylated polyalkyleneimines have a polyalkyleneimine backbone and alkoxy chains. Preferably, the polyalkyleneimine is polyethyleneimine. More preferably, the alkoxylated polyalkyleneimine is not quaternized.

If the detergent composition comprises alkoxylated polyalkyleneimines in component Z6), the composition preferably comprises from 1% to 10 wt.-%, more preferably from 1% to 8 wt.-% of alkoxylated polyalkyleneimines, based on the total weight of the detergent composition.

Preferably, the alkoxylated polyalkyleneimine used in component Z6) comprises 0.5 to 40 wt.-%, more preferably 1 to 30 wt.-%, even more preferably 2 to 20 wt.-% of the polyalkyleneimine backbone and 60 to 99 wt.-%, more preferably 60 to 95 wt.-%, even more preferably from 60 to 90 wt.-% of the alkoxy chains.

Preferably, the alkoxy chains have an average of from about 1 to about 50, more preferably from about 2 to about 40, even more preferably from about 3 to about 30 and particularly preferably from about 3 to about 20 especially from about 4 to about 15 alkoxy units, which are preferably ethoxy units. In other suitable alkoxylated polyalkyleneimines for use in component Z6), the alkoxy chains have an average of from about 0 to 30, more preferably from about 1 to about 12, even more preferably from about 1 to about 10 and particularly preferably from about 1 to about 8 propoxy units. Especially preferred are alkoxylated polyethyleneimines wherein the alkoxy chains comprise a combination of ethoxy and propoxy chains, in particular polyethyleneimines comprising chains of from 4 to 20 ethoxy units and from 0 to 6 propoxy units.

Preferably, the alkoxylated polyalkyleneimine is obtained from alkoxylation wherein the starting polyalkyleneimine has a weight-average molecular weight of from about 100 to about 60,000, preferably from about 200 to about 40,000, more preferably from about 300 to about 10,000 g/mol. In a preferred embodiment, a polyethyleneimine with a weight average molecular weight of 600 g/mol ethoxylated with 20 EO groups per NH group is used as the alkoxylated polyalkyleneimine.

Other suitable polyalkyleneimines for applicable in component Z6 of the detergent composition of the invention include compounds having the following general structure: bis((C₂H₅O)(C₂H₄O)ₙ)(CH₃)—N⁺—CₓH₂ₓ—N⁺—(CH₃)-bis((C₂H₅O)(C₂H₄O)ₙ), wherein n is a number from 20 to 30, and x is a number from 3 to 8, or sulfated or sulfonated variants thereof.

The one or more further additives Z7) are preferably selected from the group consisting of chelating agents, glass corrosion inhibitors, water, organic solvents, thickeners, foaming inhibitors, color particles, silver protecting agents, agents for preventing the tarnishing of silver, corrosion inhibitors, colorants, fillers, germicidal agents, hydrotropic agents, antioxidants, enzyme stabilizers, perfumes, solubilizers, carriers, processing aids, pigments and pH regulators.

Glass corrosion inhibitors prevent the occurrence of haze, streaks and scratches, but also iridescence of the glass surface of machine-cleaned glasses. Preferred glass corrosion inhibitors are, among others, magnesium, cobalt and zinc salts and magnesium, cobalt and zinc complexes.

Proteins and/or enzymes, such as those in Z2), may be particularly sensitive to damage such as inactivation, denaturation or degradation during storage (e.g. by physical influences, oxidation or proteolytic cleavage). In the case of microbial recovery of the proteins and/or enzymes, inhibition of the proteolysis is particularly preferred, in particular if the automatic dishwasher detergents according to the invention also contain proteases. Therefore, the detergent composition for machine dishwashing according to the invention may contain enzyme stabilizers.

Often such detergent compositions contain 0.1 to 12 wt.-%, preferably 0.2 to 10 wt.-%, more preferably 0.5 to 8 wt.-%, based on the total weight of the detergent composition, of such enzyme stabilizers.

The cleaning performance of the detergent composition according to the invention can be improved by the addition of organic solvents. Therefore, the detergent composition according to the invention may contain at least one organic solvent. Liquid machine dishwashing detergent compositions according to the invention may contain at least one organic solvent in amounts of from 0.2 to 15 wt.-%, more preferably in amounts of from 0.5 to 12 wt.-% and even more preferably in amounts of from 1.0 to 10 wt.-%, based on the total weight of the detergent composition.

These organic solvents are for example monoalcohols, diols, triols, polyols, ethers, esters and/or amides. Particular preference is given to organic solvents which are water-soluble, “water-soluble” solvents in the sense of the present application are solvents which are completely miscible with water at room temperature (i.e., without miscibility gaps). The organic solvents from the group of organic amines and/or alkanolamines are effective in terms of cleaning performance and in particular with regard to the cleaning performance of bleachable soiling, especially on tea stains.

In order to achieve the desired viscosity of a liquid detergent composition according to the invention, thickeners can be added to this composition. In the detergent composition for machine dishwashing according to the invention, the thickeners commonly used in detergent compositions for machine dishwashing can be used.

Advantageously, liquid detergent compositions of the invention contain one or more thickeners in amounts preferably from 0.1 to 8 wt.-%, more preferably from 0.2 to 6 wt.-% and even more preferably from 0.4 to 4 wt.-%, based on the total weight of the liquid detergent composition according to the invention.

Foaming inhibitors, color particles, silver protecting agents, agents for preventing the tarnishing of silver, corrosion inhibitors, colorants, fillers, germicidal agents, hydrotropic agents, antioxidants, enzyme stabilizers, perfumes, solubilizers, carriers, processing aids, pigments and pH regulators may be selected from the corresponding substances commonly used in detergent compositions for machine dishwashing.

Particularly preferably, the detergent composition for machine dishwashing according to the invention comprises
Z1) 0.1 to 15 wt.-% of component Z1),
Z2) 1×10⁻⁶ to 5 wt.-% of component Z2),
Z3) 2 to 80 wt.-% of component Z3),
Z4) 1 to 40 wt.-% of component Z4),
Z5) 0 to 15 wt.-% of component Z5),
Z6) 0 to 10 wt % of component Z6, and
Z7) 0 to 70 wt.-% of component Z7),
based on the total weight of the detergent composition.

More preferably, the detergent composition for machine dishwashing according to the invention comprises
Z1) 0.1 to 10 wt.-% of component Z1),
Z2 1×10⁻⁵ to 3 wt.-% of component Z2),
Z3) 30 to 80 wt.-% of component Z3),
Z4) 0.5 to 30 wt.-% of component Z4),
Z5) 0 to 15 wt.-% of component Z5),
Z6) 0 to 10 wt % of component Z6), and
Z7) 0 to 40 wt.-% of component Z7),
based on the total weight of the detergent composition.

Most preferably, the detergent composition for machine dishwashing according to the invention comprises Z1) 0.1 to 5 wt.-% of component Z1),
Z2) 1×10⁻⁴ to 2 wt.-% of component Z2),
Z3) 40 to 75 wt.-%, preferably 50 to 75 wt.-%, of component Z3),
Z4) 3 to 25 wt.-% of component Z4),
Z5) 0 to 15 wt.-% of component Z5),
Z6) 0 to 10 wt % of component Z6), and
Z7) 0 to 30 wt.-% of component Z7),
based on the total weight of the detergent composition.

In a further preferred embodiment, the detergent composition according to the invention contains no phosphate-based builders, and more preferably the detergent compositions according to the invention contains no phosphates, i.e. they are phosphate-free. The detergent composition for machine dishwashing according to the invention can be prepared in solid or liquid form and as a combination of solid and liquid forms.

Preference is given to detergent compositions according to the invention, which are solid at 20° C. Powder, granules, extrudates or compactates, in particular tablets in single-phase or multiphase form are particularly suitable as solid forms. Tablets are formulated for single-dose applications. The solid compositions according to the invention preferably contain less than 20 wt.-% of water, more preferably 0.1 to 20 wt.-% of water and even more preferably 0.5 to 5 wt.-%, based on the total weight of the detergent composition according to the invention. In another preferred embodiment of the invention, the detergent composition for machine dishwashing is anhydrous.

In a particularly preferred embodiment of the invention, the solid detergent composition for machine dishwashing is in the form of a tablet.

Preference is also given to detergent compositions for machine dishwashing according to the invention, which comprise component Z1) the surface active agent of formula (I), and one or more components Z2) to Z7) and are enclosed in a water-soluble foil, preferably a polyvinyl alcohol containing foil.

In the context of the present invention, the term “water soluble foil” means that the foil comprises a water-soluble polymer, copolymer or mixtures thereof in a weight fraction of at least 90 wt.-%, based on the weight of the foil. Water soluble polymers in the context of the present invention are polymers which are soluble in water at 25° C. to an amount of more than 2.5 wt.-%, based on the amount of water.

Preferable materials of the water soluble foil are at least partially composed of a substance selected from the group consisting of polyvinyl alcohols, acetalized polyvinyl alcohols, polyvinylpyrrolidones, gelatine, polyvinyl alcohols substituted with sulfate, carbonate and/or citrate, polyalkylene oxides such as polyethylene oxides, acrylamides, cellulose esters, cellulose ethers, celullose amides, cellulose, polyvinyl acetate, polycarboxylic acids and their salts, polyaminoacids or peptides, copolymers of acrylamides and (meth)acrylic acid, polysaccharides such as starch or guar derivatives, and compounds with the INCI names polyquaternium 2, polyquaternium 17, polyquaternium 18 and polyquaternium 27. In a particularly preferred embodiment, the material of the water soluble foil comprises polyvinyl alcohol.

In a further preferred embodiment of the invention, the material of the water soluble foil comprises mixtures of different substances, such as copolymers. Such mixtures enable the adjustment of the mechanical properties of the foil and the container formed thereof, and may affect the degree of water solubility. Preferably, the water soluble foil contains at least one polyvinyl alcohol and/or at least one polyvinyl alcohol copolymer.

In a further preferred embodiment, the detergent composition of the invention is liquid at 20° C. The liquid formulation, preferably based on water and/or organic solvents, can be provided in a thickened form, as a gel. Capsules (caps) are particularly suited for liquid formulations in single-phase or multiphase form. Preferably, the liquid detergent composition for machine dishwashing according to the invention contain up to 60 wt.-% of water, more preferably from 10 to 60 wt.-% of water, even more preferably 25 to 60 wt.-% of water, based on the total weight of the liquid detergent composition for machine dishwashing according to the invention.

In a particularly preferred embodiment, the detergent composition for machine dishwashing according to the invention is provided in the form of a powder, a tablet, a gel, a pod, a cap or as a liquid rinse aid.

Another further aspect of the invention is a method of cleaning dishes in a dishwashing machine, wherein contaminated dishes are treated in the dishwashing machine with an aqueous alkaline composition comprising the detergent composition for machine dishwashing according to the invention.

In this method of cleaning dishes, the pH of the aqueous alkaline solution is preferably 8 or above, more preferably 9 or above. In a particularly preferred embodiment, the pH of the aqueous alkaline solution is from 8 to 13. In a particularly more preferred embodiment, the pH of the aqueous alkaline solution is from 9 to 12.

The invention is explained in more detail below by the examples and the claims. Unless explicitly stated otherwise in the examples, the percentages in the examples are to be understood as percent by weight (wt.-%).

### EXAMPLES

### Example 1

Preparation of levulinic acid (LVA) lauryl alcohol ethoxylate ester—C12/14 (EO) 5 LVA. To a 100 mL three-necked flask equipped with mechanical stirrer, Dean-Stark receiver and N₂ inlet, 64.6 g (0.160 mol) lauryl alcohol ethoxylate (lauryl alcohol+5 mol ethylene oxide (EO), Clariant Produkte Deutschland GmbH) and 18.6 g levulinic acid (0.155 mol, 97% pure) were charged under nitrogen atmosphere. 0.66 g phosphonic acid (0.0040 mol, 50% in water) were added and the mixture heated to 180° C. and the water of condensation was distilled off from the equilibrium. After about 32 h, the levulinate ester was obtained in as a light yellow, clear liquid (acid number: 4.6 mgKOH/g, saponification number: 120.3 mgKOH/g).

### Example 2

Preparation of levulinic acid lauryl alcohol ethoxylate ester-C12/14 (EO) 8 LVA. To a 100 mL three-necked flask equipped with mechanical stirrer, Dean-Stark receiver and N₂ inlet, 70.0 g (0.133 mol) lauryl alcohol ethoxylate (lauryl alcohol+8 mol EO, Clariant Produkte Deutschland GmbH) and 14.4 g levulinic acid (0.124 mol, 97% pure) were charged under nitrogen atmosphere. 0.41 g phosphonic acid (0.0035 mol, 50% in water) were added and the mixture heated to 180° C. and the water of condensation was distilled off from the equilibrium. After about 32 h, the levulinate ester was obtained in as a light yellow, clear liquid (acid number: 4.6 mgKOH/g, saponification number: 95.4 mgKOH/g).

### Example 3

Preparation of levulinic acid oleyl alcohol ethoxylate ester-Oleyl (EO) 6 LVA. To a 100 mL three-necked flask equipped with mechanical stirrer, Dean-Stark receiver and N₂ inlet, 67.2 g (0.129 mol) oleyl alcohol ethoxylate (oleyl alcohol+5 mol EO, Clariant Produkte Deutschland GmbH) and 15.0 g levulinic acid (0.125 mol, 97% pure) were charged under nitrogen atmosphere. 0.38 g phosphonic acid (0.0033 mol, 50% in water) were added and the mixture heated to 180° C. and the water of condensation was distilled off from the equilibrium. After about 35 h, the levulinate ester was obtained in as a light yellow, slightly turbid liquid (acid number: 3.3 mgKOH/g, saponification number: 89.1 mgKOH/g).

### Example 4

Preparation of levulinic acid oleyl alcohol ethoxylate ester-Oleyl (EO) 10 LVA. To a 100 mL three-necked flask equipped with mechanical stirrer, Dean-Stark receiver and N₂ inlet, 70.0 g (0.096 mol) oleyl alcohol ethoxylate (oleyl alcohol+10 mol EO, Clariant Produkte Deutschland GmbH) and 10.8 g levulinic acid (0.093 mol, 97% pure) were charged under nitrogen atmosphere. 0.29 g phosphonic acid (0.0029 mol, 50% in water) were added and the mixture heated to 180° C. and the water of condensation was distilled off from the equilibrium. After about 35 h, the levulinate ester was obtained in as a light yellow, clear liquid (acid number: 3.8 mgKOH/g, saponification number: 70.5 mgKOH/g).

### Example 5

Preparation of levulinic acid cocoyl alcohol ethoxylate ester-Cocoyl (EO) 5 LVA. To a 100 mL three-necked flask equipped with mechanical stirrer, Dean-Stark receiver and N₂ inlet, 58.2 g (0.140 mol) Genapol® C 050 (ethoxylated cocoyl alcohol, Clariant Produkte Deutschland GmbH) and 16.3 g levulinic acid (0.136 mol, 97% pure) were charged under nitrogen atmosphere. 0.57 g phosphonic acid (0.0035 mol, 50% in water) were added and the mixture heated to 180° C. and the water of condensation was distilled off from the equilibrium. After about 30 h, the levulinate ester was obtained in 99% yield as a light yellow, clear liquid (acid number: 2.9 mgKOH/g, saponification number: 118.4).

### Example 6

Alternative preparation of levulinic acid cocoyl alcohol ethoxylate ester-Cocoyl (EO) 5 LVA.

To a 250 mL three-necked flask equipped with magnetic stirrer, Dean-Stark receiver, vacuum and N₂ inlet, 145.5 g (0.35 mol) Genapol C 050 (ethoxylated fatty alcohol, Clariant Produkte Deutschland GmbH) and 53.1 g methyl levulinate (0.40 mol, 98% pure) were charged under nitrogen atmosphere. 0.04 g Titanium (IV) butoxide (200 ppm) were added and the mixture heated to 130° C. Subsequently, vacuum was applied (250 mbar). After about 16 h, the pressure was reduced to 30 mbar to distill off excess methyl levulinate. The levulinic acid Genapol C 050 ester was obtained as a brown, clear liquid (saponification number 116.1 mgKOH/g).

### Example 7

Rinse aiding performance of detergent compositions for machine dishwashing

The rinse aiding performance of the detergent compositions F4 to F6 according to the invention was investigated. As comparative examples, the rinse aiding performance of the comparative detergent compositions F1 (without surface active agent), F2 and F3 (with different standard EO/PO-surface active agents) was tested.

### Testing Conditions:

Dishwashing machine: Miele G 1222 SC GSL2
Testware dishes: 12 drinking glasses
(8 material groups) 3 PP bowls
   3 melamine plates
   3 butter dishes
   4 knives (stainless steel; lower quality)
   4 knives (stainless steel; higher quality)
   3 porcelain plates (medium quality)
   3 porcelain plates (lower quality)
Dishwashing program: program “R^{50/3} min/KI65”
Water hardness: 21+/−1°dH
Water softening: none
Detergent dosage: 18 g, added to the dosage chamber before starting the test
Contamination: 100 g frozen dirt, added immediately after the opening of the dosage chamber
Rinse aid: none
Cleaning cycles: 4

All testware dishes were treated once with demineralized water, Neodisher A 8, citric acid and again demineralized water.

### Evaluation:

Evaluation of the testware was started at least 60 minutes after opening the door of the dishwashing machine after completion of the dishwashing cycle. For each test, dishwashing cycles 2 to 4 were evaluated. The assessment was carried out according to the following rating:

### Rinse Aid Effects Considered for the Visual Rating:

Spotting concentrated punctional residue
Contact spots Stains resultant from contact points between the testware dishes and parts of the dishwashing machine
Stripes Rinse aid stripes
Film formation continuous film spread uniformly on the testware dishes
Structured film formation Dispersed torn film
Solid residues Solid powder or crystalline residues
Fatty residues Fatty drops or fatty film formation
Iridescence Shimmering, iridescence
Other other effects

### Visual Rating Marks:

10 Perfect
9 Perfect to barely visible
8 Barely visible
7 Barely visible to visible
6 Visible
5 Visible to disturbing
4 Disturbing
3 Disturbing to unacceptable
2 Unacceptable
1 Absolutely unacceptable

The combination of the above listed eight rinse aid effects leads to a rating from 1 to 10 according to the above visual rating marks, wherein a rating of 1 represents the worst performance and a rating of 10 represents the best performance. For each of the above material groups (glass, plastics, porcelain, stainless steel) an average rating for the dishwashing cycles 2 to 4 was determined. The average of the visual assessment is then multiplied by 10 in order to calculate an average respective performance index for each material group (10: worst performance; 100: best performance).

### Compositions:

The detergent compositions of examples F4 to F6 (according to the invention) and of comparative examples F1 (without surface active agent), F2 and F3 (with different standard EO/PO-surface active agent) are shown in Table 1. The results are also shown in Table 1.

TABLE 1
Detergent compositions and rinse aiding performance

| | Example | | | | | |
|---|---|---|---|---|---|---|
| | F1 (cp.) | F2 (cp.) | F3 (cp.) | F4 | F5 | F6 |
| Component | wt.-%*⁾ | wt.-%*⁾ | wt.-%*⁾ | wt.-%*⁾ | wt.-%*⁾ | wt.-%*⁾ |
| trisodium citrate | 30.0 | 30.0 | 30.0 | 30.0 | 30.0 | 30.0 |
| dihydrate | | | | | | |
| sodium carbonate | 35.0 | 35.0 | 35.0 | 35.0 | 35.0 | 35.0 |
| sodium silicate | 2.0 | 2.0 | 2.0 | 2.0 | 2.0 | 2.0 |
| MGDA-Na₃ | 0.4 | 0.4 | 0.4 | 0.4 | 0.4 | 0.4 |
| Sodium hydrogen | 1.3 | 1.3 | 1.3 | 1.3 | 1.3 | 1.3 |
| carbonate | | | | | | |
| polycarboxylate | 5.0 | 5.0 | 5.0 | 5.0 | 5.0 | 5.0 |
| sodium percarbonate | 14.0 | 14.0 | 14.0 | 14.0 | 14.0 | 14.0 |
| TAED | 2.0 | 2.0 | 2.0 | 2.0 | 2.0 | 2.0 |
| HEDP | 1.5 | 1.5 | 1.5 | 1.5 | 1.5 | 1.5 |
| protease | 0.4 | 0.4 | 0.4 | 0.4 | 0.4 | 0.4 |
| amylase | 0.3 | 0.3 | 0.3 | 0.3 | 0.3 | 0.3 |
| C12/14-(EO)8-(PO)4 | — | 4.0 | — | — | — | — |
| C12/14-(EO)6-(PO)4 | — | — | 4.0 | | — | — |
| Oleyl-(EO)6-LVA | — | — | — | 4.0 | — | — |
| Oleyl-(EO)10-LVA | — | — | — | | 4.0 | — |
| Lauryl-(EO)8-LVA | — | — | — | — | — | 4.0 |
| sodium sulfate**⁾ | 7.3 | 3.3 | 3.3 | 3.3 | 3.3 | 3.3 |
| Performance | 26 | 48 | 58 | 38 | 51 | 42 |
| index on glass | | | | | | |
| Performance | 63 | 61 | 61 | 58 | 59 | 60 |
| index on plastics | | | | | | |
| Performance | 54 | 65 | 69 | 66 | 73 | 65 |
| index on porcelain | | | | | | |
| Performance | 47 | 48 | 54 | 50 | 63 | 37 |
| index on stainless | | | | | | |
| steel | | | | | | |
| Sum Performance | 190 | 222 | 242 | 212 | 246 | 204 |
| Index all materials | | | | | | |

*⁾The ingredients were added according to their active component content in wt.-%.
**⁾Sodium sulfate is added as a filler for a constant mass balance of the detergent composition, without a function and without influence on the performance of the detergent composition.

All compositions F2 to F6 show superior rinse aiding performance compared to the composition F1 (without surface active agent).

By using a levulinic ester endcapped surface active agent according to the invention (cp. examples F4 to F6) as surface active agent ingredient equal or even better rinse aiding performance can be achieved compared to composition F2 and F3 (both with a standard EO/PO-surface active agent).

These results prove the suitability of levulinic esters as rinse aiding ingredient in automatic dishwashing compositions.

### Example 7

### Application Test

The foaming behavior of the previously described levulinic acid esters was tested in a shake foam test in aqueous formulation according to the following recipe:

0.5% weight in water, distilled. The mixture is homogenized by stirring with a magnetic stirrer and the pH is confirmed to be within 5 to 7. A graduated 100 mL cylinder is charged with 20 mL of the solution carefully in a way that avoids foam formation. The cylinder is capped and shaken ten times vertically. The time after the shaking process is measured and foam volume is recorded. Initial foam volume is observed at t=0 min. For comparison, the foaming of C12/14-(EO) 6-(PO) 4 (Genapol EP 2464, Clariant Produkte Deutschland GmbH), a commercial low-foaming surfactant and rinse aid was assessed. The results are summarized in Table 2. The levulinic acid esters according to the invention show equal or superior foaming properties with reduced flash foam.

TABLE 2
Foam measurements in a graduated 100 mL cylinder

| | Active ingredient (0.5% in water, dist.) | | | |
|---|---|---|---|---|
| | C12/14- | C12/14 | Cocoyl | Oleyl- |
| | (EO)6-(PO)4 | (EO)5-LVA | (EO)5-LVA | (EO)6-LVA |
| Foam, 0 min | 50 | 30 | 22 | 25 |
| Foam, 5 min | 26 | 23 | 21 | 25 |
| Foam, 30 min | 22 | 22 | 21 | 24 |
| Foam, 45 min | 22 | 22 | 20 | 24 |

As shown by the examples the end group-capped surface active agents of formula (I) according to the invention have a good foam-suppressing action.

Moreover, they are sufficiently stable even in mildly alkaline environment over the usual periods of use.

Compared to the alkyl end-capped nonionic surfactants of the prior art (e.g. such as described in DE 37 44 525 C1), the production of the end group-capped surface active agents of formula (I) according to the invention is not associated with the inevitable occurrence of salts in the Williamson ether synthesis.

Furthermore, the surface active agents of the formula (I) according to the invention have a good biodegradability and low toxicity.

1-18. (canceled)

19. A surface-active agent of formula (I)
R¹—O [symbol] (CH₂CH₂O)ₓ(CHR³CHR⁴O)_{y} [symbol] C(O)—Z—C(O)—R² (I)
wherein
R¹ is a linear or branched alkyl group having 8 to 30 carbon atoms, or a linear or branched alkenyl group having one or more double bonds and 8 to 30 carbon atoms;
R² is a linear or branched alkyl group having 1 to 3 carbon atoms;
R³, R⁴ are chosen such that either R³ or R⁴ is hydrogen and the other is a methyl group;
Z is a linear or branched alkylene having 1 to 3 carbon atoms;
X is an integer from 1 to 25; and
y is an integer from 0 to 10,
wherein x is greater than y and if y is ≠0, the structure units-CH₂CH₂O— and —CHR³CHR⁴O— can be arranged in any order.

20. The surface-active agent according to claim 19, wherein
R¹ is a linear or branched alkyl group having 8 to 22 carbon atoms, or a linear or branched alkenyl group having one or more double bonds and 8 to 22 carbon atoms;
X is an integer from 2 to 20; and
y is an integer from 0 to 5,
wherein if y is ≠0, the ratio of x to y is at least 2.

21. The surface-active agent according to claim 19, wherein
R¹ is a linear or branched alkyl group having 8 to 18 carbon atoms, or a linear or branched alkenyl group having one or more double bonds and 8 to 18 carbon atoms;
R² is methyl;
Z is ethylene (—CH₂—CH₂—);
X is an integer of from 2 to 15; and
y is 0.

22. A process for the preparation of the surface-active agent according to claim 19, wherein
a compound of formula (II)
R¹—O [symbol] (CH₂CH₂O)ₓ(CHR³CHR⁴O)_{y} [symbol] —H (II)
is esterified in presence of at least one acidic esterification catalyst or is transesterified in presence of at least one transesterification catalyst with a compound of formula (III)
R⁵⁰—C(O)—Z—C(O)—R² (III),
wherein in the formulae, R¹, R², R³, R⁴, x, y and Z are as defined in claim 19, and R⁵ denotes a C₁ to C₄-alkyl group or hydrogen (H).

23. The process according to claim 22, wherein the compound of formula (III) is levulinic acid or a levulinic acid C₁ to C₄-alkyl ester.

24. The process according to claim 22, wherein the compound of formula (II) is obtained by the addition of ethylene oxide and optionally propylene oxide to a primary alcohol R¹OH, wherein the ethylene oxide is a bio-ethylene oxide obtained from renewable resources and the optional propylene oxide is a bio-propylene oxide obtained from renewable resources.

25. The process according to claim 22, wherein the compound of formula (III) is bio-based and produced from second generation feedstocks.

26. The process according to claim 22, wherein the at least one acidic esterification catalyst is selected from the group consisting of mineral acids, sulfonic acids, and acidic ion exchangers.

27. The process according to claim 22, wherein the at least one transesterification catalyst is a Lewis acid based on titanium, aluminum, zinc, and/or tin.

28. The process according to claim 22, wherein esterification is carried out at temperatures from 80° C. to 230° C. and the water of condensation is continuously removed from the equilibrium.

29. The process according to claim 22, wherein transesterification is carried out at temperatures from 80° C. to 230° C. and condensation alcohol of the transesterification is continuously removed from the equilibrium.

30. A method of regulating and minimizing foam, wherein the method comprises the step of adding at least one surface-active agent according to claim 19 to a detergent, an industrial cleaner, or a formulation for metal working, textile processing, crop protection, or pigment dispersion.

31. A method of improving the rinse aiding properties and/or the drying capacity of a machine dishwashing detergent composition, wherein the method comprises the step of adding at least one surface-active agent according to claim 19 to the machine dishwashing detergent composition.

32. A detergent composition for machine dishwashing, wherein the detergent composition comprises at least one surface-active agent according to claim 19.

33. The detergent composition according to claim 32, wherein the detergent composition comprises from 0.1 wt.-% to 15 wt.-% of the at least one surface-active agent according to claim 19.

34. The detergent composition according to claim 32 comprising:
Z1) at least one surface-active agent according to claim 19, and one or more further components selected from Z2) to Z7):
Z2) one or more enzymes selected from the group consisting of proteases, amylases, lipases, hemicellulases, cellulases, perhydrolases, and oxidoreductases;
Z3) one or more builders;
Z4) one or more bleaching agents;
Z5) one or more surfactants;
Z6) one or more polymers; and
Z7) one or more further additives selected from the group consisting of chelating agents, glass corrosion inhibitors, water, organic solvents, thickeners, foaming inhibitors, color particles, silver protecting agents, agents for preventing the tarnishing of silver, corrosion inhibitors, colorants, fillers, germicidal agents, hydrotropic agents, antioxidants, enzyme stabilizers, perfumes, solubilizers, carriers, processing aids, pigments, and pH regulators.

35. The detergent composition according to claim 32, wherein the detergent composition is a powder, tablet, gel, pod, cap, or liquid rinse aid.
